(19) United States
(12) Patent Application Publication
(10) Pub. No.: US 2010/0234648 A1
(43) Pub. Date: Sep. 16, 2010

(54) PROCESS FOR PRODUCING TERTIARY OLEFIN AND ALIPHATIC ALCOHOL

(75) Inventors: Masanobu Yamase, Chiba (JP); Yoshiaki Suzuki, Chiba (JP); Kazuya Yoshikawa, Chiba (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, Chuo-ku, Tokyo (JP)

(21) Appl. No.: 12/294,445

(22) PCT Filed: Mar. 29, 2007
(86) PCT No.: PCT/JP2007/057608

§ 371 Date: Sep. 25, 2008

(30) Foreign Application Priority Data
Mar. 31, 2006 (JP) 2006-097939

(51) Int. Cl. C07C 41/00

(52) U.S. Cl. 568/697

(57) ABSTRACT

A process for producing a tertiary olefin and an aliphatic alcohol, which comprises adding an aliphatic alcohol to an alkyl tertiary-alkyl ether in advance, and then, decomposing the alkyl tertiary-alkyl ether. Examples of the aliphatic alcohol include methanol, ethanol and the like.

### TECHNICAL FIELD

The present invention relates to a process for producing a tertiary olefin and an aliphatic alcohol. More particularly, it relates to a process for producing a tertiary olefin and an aliphatic alcohol by decomposing an alkyl tertiary-alkyl ether into a tertiary olefin and an aliphatic alcohol in gas-phase, and separating each of the resultant tertiary olefin and the resultant aliphatic alcohol, thereby recovering the tertiary olefin and the aliphatic alcohol individually, characterized by being capable of suppressing the formation of tar-like substances during a period of time from the evaporation of the alkyl tertiary-alkyl ether to the decomposition thereof, thereby eliminating problematic blockages of equipments and pipes and achieving a long-term continuous stable operation of plants.

### BACKGROUND ART

The processes for producing tertiary olefins and aliphatic alcohols by using alkyl tertiary-alkyl ethers as a raw material are known. With respect to these methods, for example, JP-A-59-88431 provides a method for solving the problems such as decrease in the activity of solid acid catalysts due to the deposition of diisobutylene, suppression of the production of dimethyl ether due to the limited concentration of methanol, and loss of methanol; and JP-A-2003-2852 provides a method for solving the problems such as suppression of incorporation of water and tertiary-butanol into methanol, and loss of methanol and isobutylene. However, these do not provide a method for solving a following problem: when alkyl tertiary-alkyl ethers are heated and vaporized for the decomposition thereof, tar-like substances are formed and deposited in equipments and pipes, thereby causing the blockages of equipments and pipes and the difficulty in long-term continuous operation of plants. Accordingly, there is a demand for a method for solving said problem.

### DISCLOSURE OF INVENTION

An object of the present invention is to provide a process for producing a tertiary olefin and an aliphatic alcohol by decomposing an alkyl tertiary-alkyl ether into a tertiary olefin and an aliphatic alcohol in gas-phase, and separating each of the resultant tertiary olefin and the resultant aliphatic alcohol, thereby recovering the tertiary olefin and the aliphatic alcohol individually, characterized by being capable of suppressing the formation of tar-like substances during a period of time from the evaporation of the alkyl tertiary-alkyl ether to the decomposition thereof, thereby eliminating problematic blockages of equipments and pipes and achieving a long-term continuous stable operation of plants.

Namely, the present invention relates to a process for producing a tertiary olefin and an aliphatic alcohol, which comprises decomposing an alkyl tertiary-alkyl ether into a tertiary olefin and an aliphatic alcohol in gas-phase, and separating each of the resultant tertiary olefin and the resultant aliphatic alcohol, thereby recovering the tertiary olefin and the aliphatic alcohol individually, wherein the process comprises adding an aliphatic alcohol to the alkyl tertiary-alkyl ether in advance such that the content of aliphatic alcohol is from 2 to 10% by weight per 100% by weight of the alkyl tertiary-alkyl ether, and then, decomposing the alkyl tertiary-alkyl ether.

### BRIEF DESCRIPTION OF DRAWINGS

FIG. 1 is a schematic diagram showing an example of the process of the present invention comprising decomposing an alkyl tertiary-alkyl ether, and separating each of the resultant tertiary olefin and the resultant aliphatic alcohol, thereby recovering the tertiary olefin and the aliphatic alcohol individually.

FIG. 2 is a graph based on the results of Example 1 and Comparative Example 1 as shown in Table 1.

### EXPLANATION OF REFERENCE NUMERALS

1. Alkyl tertiary-alkyl ether, 2. Aliphatic alcohol, 3. Pump, 4. Evaporator, 5. Waste heat recovery equipment, 6. Preheater, 7. Decomposition reactor, 8. Separation step, 9. Extraction step, 10. Water, 11. Tertiary olefin (product), 12. Purification step, 13. Aliphatic alcohol (product), 14. Waste water

### MODE FOR CARRYING OUT THE INVENTION

Examples of the alkyl tertiary-alkyl ether to be used as a raw material in the present invention include methyl tertiary-butyl ether, ethyl tertiary-butyl ether, propyl tertiary-butyl ether and the like.

The alkyl tertiary-alkyl ether to be used in the present invention is generally produced by using a distillation fraction (spent BB), which is a residue left after the extraction of butadiene from a C4 hydrocarbon mixture obtained by steam cracking of naphtha, as a raw material. Examples of the spent BB include a mixture containing, as a main component, a C4 distillation fraction containing from 30 to 50% by weight of isobutylene, from 10 to 40% by weight of 1-butene, from 10 to 20% by weight of 2-butene, from 10 to 20% by weight of n-butane, from 1 to 10% by weight of isobutane, from 0.1 to 2% by weight of 1,3-butadiene and from 0.01 to 0.2% by weight of propadiene. The mixture generally contains from several ppm by weight to several hundred ppm by weight of acetaldehyde.

As the alkyl tertiary-alkyl ether produced by using the spent BB as a raw material, methyl tertiary-butyl ether (MTBE) is generally selected. A commercially available MTBE generally contains from 1 ppm by weight to 0.5% by weight of methanol.

In view of the composition of the spent BB, the formation mechanism of tar-like substances may involve polycondensation reaction of aldehydes, polymerization reaction of dienes, and C—C bond formation reaction such as carbonylene reaction (Prins reaction) and the like. Thus, the present inventors have performed various tests, and finally found the suppressing effect of aliphatic alcohol on the formation of tar-like substances in the present invention.

For the purpose of obtaining isobutylene as a tertiary olefin and methanol as an aliphatic alcohol, MTBE is preferably used as an alkyl tertiary-alkyl ether to be decomposed in gas-phase in the present invention.

Examples of the aliphatic alcohol in the present invention include methanol, ethanol and the like. Among them, preferred is methanol, since a commercially available MTBE generally contains from 1 ppm by weight to 0.5% by weight of methanol, as described above, and methanol is generated by the decomposition of MTBE.

The method for decomposing the alkyl tertiary-alkyl ether is not particularly limited, but includes, for example, a method comprising charging an aluminum-containing silica catalyst as a catalyst into a reactor, and passing an alkyl tertiary-alkyl ether through this reactor, thereby decomposing the alkyl tertiary-alkyl ether.

The method for separating each of a tertiary olefin and an aliphatic alcohol, both of which are obtained by the decomposition, and recovering the tertiary olefin and the aliphatic alcohol individually may include a known method, for example, a method comprising (i) separating each of a tertiary olefin containing a small amount of an azeotropic aliphatic alcohol and a distillation fraction mainly consisting of an aliphatic alcohol by distillation, (ii) extracting the azeotropic aliphatic alcohol in the tertiary olefin with water, thereby obtaining the tertiary olefin, and (iii) separating a mixture of the distillation fraction mainly consisting of an aliphatic alcohol and the azeotropic aliphatic alcohol extracted with water into the aliphatic alcohol and waste water by distillation, thereby obtaining the aliphatic alcohol.

For the purpose of producing a tertiary olefin and an aliphatic alcohol by the decomposition of the alkyl tertiary-alkyl ether, an aliphatic alcohol is added to the alkyl tertiary-alkyl ether prior to the decomposition thereof in the present invention. The aliphatic alcohol is added such that the content of aliphatic alcohol after the addition thereof is from 2 to 10% by weight, preferably from 2 to 7% by weight, per 100% by weight of the alkyl tertiary-alkyl ether. When the content of aliphatic alcohol is less than 2% by weight, a suppressing effect on the formation of tar-like substances may not be sufficiently obtained. When the content exceeds 10% by weight, an amount to be treated in a subsequent step is increased, thereby causing economic disadvantages.

The method and the site for adding the aliphatic alcohol to the alkyl tertiary-alkyl ether are not particularly limited as long as a homogeneous mixing of the alkyl tertiary-alkyl ether and the aliphatic alcohol is attained. Examples of the method include a method containing feeding an alkyl tertiary-alkyl ether together with an aliphatic alcohol to a pump for pressurizing the alkyl tertiary-alkyl ether, by joining a pipe for feeding the aliphatic alcohol to a pipe for transporting the alkyl tertiary-alkyl ether in the inlet side of the pump. In addition, the same effect is produced by increasing a ratio of an aliphatic alcohol to be used to a tertiary olefin in the synthesis step of an alkyl tertiary-alkyl ether. In this case, however, it is difficult to separate a large amount of unreacted aliphatic alcohol, which is left after the synthesis step of the alkyl tertiary-alkyl ether, in the separation step. Therefore, the site for adding the aliphatic alcohol is preferably in upstream of an evaporator to be used in the decomposition step of the alkyl tertiary-alkyl ether.

The addition of the aliphatic alcohol to the alkyl tertiary-alkyl ether may be conducted intermittently, but is preferably conducted continuously in order to keep the concentration of aliphatic alcohol in the alkyl tertiary-alkyl ether constant and to suppress more stably the formation of tar-like substances.

A part of an aliphatic alcohol obtained by the decomposition of the alkyl tertiary-alkyl ether may be recycled and used as the aliphatic alcohol to be added to the alkyl tertiary-alkyl ether. For example, when MTBE synthesized by using a spent BB and methanol as raw materials is decomposed to obtain isobutylene and methanol, the added methanol is preferably used for the synthesis of MTBE together with methanol obtained from the decomposition product.

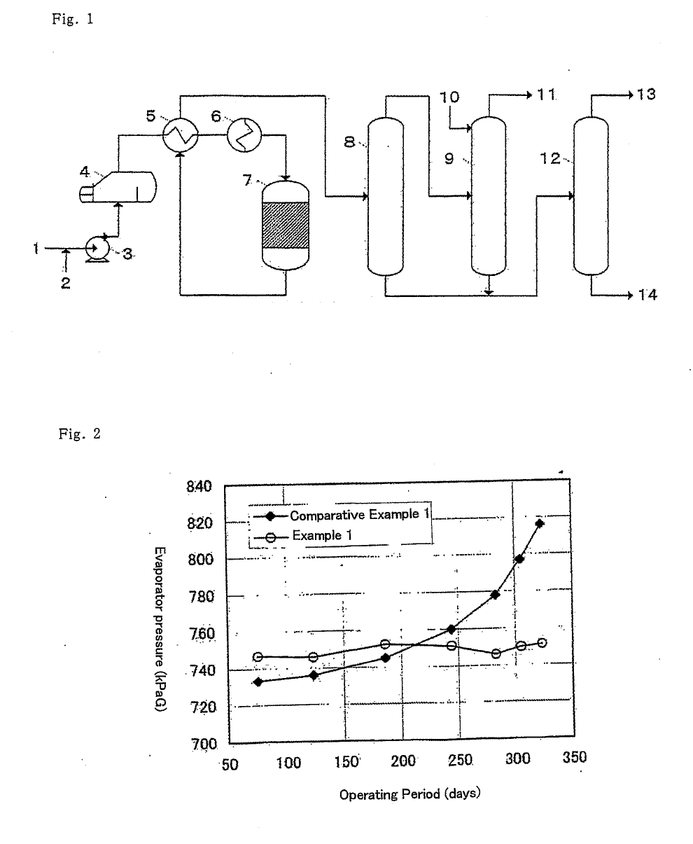

FIG. 1 is a schematic diagram showing an example of the process comprising decomposing an alkyl tertiary-alkyl ether, and separating each of the resultant tertiary olefin and the resultant aliphatic alcohol, thereby recovering the tertiary olefin and the aliphatic alcohol individually.

An alkyl tertiary-alkyl ether (1) and an aliphatic alcohol (2) are mixed and fed to a pump (3) by joining a pipe for feeding the aliphatic alcohol (2) to a pipe for transporting the alkyl tertiary-alkyl ether (1). The alkyl tertiary-alkyl ether mixed with the aliphatic alcohol is vaporized by the evaporator (4), and heated by a waste heat recovery equipment (5) and a preheater (6). Then, the heated alkyl tertiary-alkyl ether is fed to a decomposition reactor (7) and therein decomposed into a tertiary olefin and an aliphatic alcohol. The resultant tertiary olefin and the resultant aliphatic alcohol are cooled by a waste heat recovery equipment (5), and separated individually in a separation step (8). A small amount of the aliphatic alcohol contained in the tertiary olefin is extracted with water (10) to be supplied in an extraction step (9), thereby obtaining the tertiary olefin (product) (11). Meanwhile, both of the aliphatic alcohol separated in the separation step (8) and the aliphatic alcohol extracted with water in the extraction step (9) are fed to a purification step (12), wherein waste water (14) is separated, thereby obtaining the aliphatic alcohol (product) (13).

### EXAMPLES

The present invention will be illustrated in more detail with reference to the following examples.

### Example 1

According to a process as shown in FIG. 1, isobutylene and methanol were produced by using MTBE containing from about 0.5 to 0.9% by weight of methanol as a raw material. Methanol was continuously added to MTBE such that the content of methanol, including one originated from the above-mentioned raw material, was not less than 2% by weight per 100% by weight of MTBE. Then, MTBE was vaporized by an evaporator, and heated by a waste heat recovery equipment and a preheater. Then, the heated MTBE was fed to a decomposition reactor charged with an aluminum-containing silica catalyst, and therein decomposed. Both of isobutylene and methanol obtained by the decomposition of MTBE were cooled by a waste heat recovery equipment, and separated individually in a first distillation column. By extracting a small amount of methanol contained in isobutylene obtained from the top of the first distillation column with water in an extraction column, isobutylene (product) was obtained at the top of the extraction column. Then, methanol obtained from the bottom of the first distillation column and the methanol-water mixture obtained from the bottom of the extraction column were subjected to the separation treatment in a second distillation column, thereby obtaining methanol (product) at the top of the second distillation column and waste water at the bottom of the column.

A change in a pressure of the evaporator over time was monitored to determine the blockage states of equipments and pipes in downstream of the evaporator. The operation was carried out for 325 days. The pressure was increased by about 5 kPaG during from 76 to 323 operating days, due to the deposition of tar-like substances. After opening and checking the equipments and the pipes, it was found that only a small amount of tar-like substances was deposited therein.

### Comparative Example 1

Example 1 was repeated except that the addition of methanol according to the present invention was not performed. The operation was carried out for 365 days. The pressure of the evaporator was increased by about 80 kPaG during from 76 to 323 operating days, due to the deposition of tar-like substances. After opening and checking the equipments and the pipes, it was observed that a large amount of tar-like substances was deposited in each part of them. The results are shown in Table 1.

The results of the changes in the evaporator pressure over operating days, as shown in Table 1, are shown graphically in FIG. 2. While the evaporator pressure in Example 1 was not substantially changed, the pressure in Comparative Example 1 was dramatically increased after about 250 operating days. It was found that the addition of methanol according to the present invention was necessary for a long-term continuous operation, as shown in Example 1.

TABLE 1

| | Example 1 | | Comparative Example 1 | |
|---|---|---|---|---|
| Operating | Evaporator | Content of | Evaporator | Content of |
| days | pressure | methanol | pressure | methanol |
| (days) | (kPaG) | (% by weight) | (kPaG) | (% by weight) |
| 76 | 747 | 2.67 | 733 | — |
| 123 | 746 | — | 736 | 0.51 |
| 186 | 753 | — | 745 | 0.87 |
| 244 | 751 | 2.50 | 760 | — |
| 283 | 746 | 2.56 | 778 | — |
| 305 | 750 | — | 797 | 0.69 |
| 323 | 751 | — | 816 | — |

Note:
The “—” as shown in Table 1 means that the content of methanol was not determined. In this regard, MTBE used as a raw material contains about from 0.5 to 0.9% by weight. In Example 1, methanol was continuously added such that the total content of methanol was not less than 2% by weight per 100% by weight of MTBE, whereas methanol was not added in Comparative Example 1.

### INDUSTRIAL APPLICABILITY

According to the present invention, a process for producing a tertiary olefin and an aliphatic alcohol by decomposing an alkyl tertiary-alkyl ether into a tertiary olefin and an aliphatic alcohol in gas-phase, and separating each of the resultant tertiary olefin and the resultant aliphatic alcohol, thereby recovering the tertiary olefin and the aliphatic alcohol individually, characterized by being capable of suppressing the formation of tar-like substances during a period of time from the evaporation of the alkyl tertiary-alkyl ether to the decomposition thereof, thereby eliminating problematic blockages of equipments and pipes and achieving a long-term continuous stable operation of plants, is provided.

1. A process for producing a tertiary olefin and an aliphatic alcohol, which comprises decomposing an alkyl tertiary-alkyl ether into a tertiary olefin and an aliphatic alcohol in gas-phase, and separating each of the resultant tertiary olefin and the resultant aliphatic alcohol, thereby recovering the tertiary olefin and the aliphatic alcohol individually, wherein the process comprises adding an aliphatic alcohol to the alkyl tertiary-alkyl ether in advance such that the content of aliphatic alcohol is from 2 to 10% by weight per 100% by weight of the alkyl tertiary-alkyl ether, and then, decomposing the alkyl tertiary-alkyl ether.

2. The process according to claim 1, wherein the content of aliphatic alcohol is from 2 to 7% by weight per 100% by weight of the alkyl tertiary-alkyl ether.

3. The process according to claim 1, wherein a part of the aliphatic alcohol generated by decomposing the alkyl tertiary-alkyl ether is recycled and used as an aliphatic alcohol to be added to the alkyl tertiary-alkyl ether.

4. The process according to claim 1, wherein the alkyl tertiary-alkyl ether is vaporized by an evaporator, and then, is decomposed in gas-phase.

5. The process according to claim 4, wherein the aliphatic alcohol is added in upstream of the evaporator.
